(19) United States
(12) Patent Application Publication
(10) Pub. No.: US 2005/0027018 A1
(43) Pub. Date: Feb. 3, 2005

(54) Polymorphs of butylated hydroxy anisole

(75) Inventor: Julius Remenar, Framingham, MA (US)

Correspondence Address: TRANSFORM PHARMACEUTICALS, INC., 29 HARTWELL AVENUE, LEXINGTON, MA, 02421, US

(73) Assignee: Transform Pharmaceuticals, Inc., Lexington, MA (US)

(21) Appl. No.: 10/902,658

(22) Filed: Jul. 29, 2004

Related U.S. Application Data
(60) Provisional application No. 60/492,135, filed on Aug. 1, 2003.
(60) Provisional application No. 60/505,627, filed on Sep. 24, 2003.

(51) Int. Cl. A61K 31/075

(52) U.S. Cl. 514/718

(57) ABSTRACT

A novel polymorph of butylated hydroxy anisole (BHA) and techniques for the enhancement of the antioxidant properties of BHA are discussed.

### RELATED APPLICATIONS

This application claims benefit to U.S. Provisional Application No. 60/492,135 filed on Aug. 1, 2003 and U.S. Provisional Application No. 60/505,627 filed on Sep. 24, 2003, the contents of which are incorporated herein by reference in their entirety.

### FIELD OF THE INVENTION

The present invention relates to common antioxidants, and methods for preparing same.

### BACKGROUND OF THE INVENTION

Many products naturally contain or are produced with materials comprising oxidizable groups. These materials are relatively unstable during storage and degrade over time. Thus, products having increased storage stability are strongly desired. Bearing in mind the problems and deficiencies of the prior art, it is therefore desirable to provide a chemically stable product which comprises one or more oxidation susceptible moieties.

Butylated hydroxy anisole (BHA) (2(3)-tert-butyl-4-methoxy-phenol) is a common and effective antioxidant. BHA is normally commercially available as a mixture of two isomers, 2-tert-butyl-4-methoxy-phenol and 3-tert-butyl-4-methoxy-phenol, due to its synthetic pathway. The former is the major isomer in the commercially available mixture, and is represented by the structure (I):

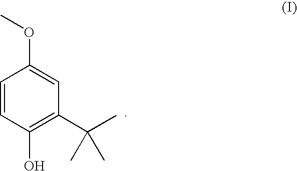

One of the drawbacks of this compound is its less than desirable reliability as an antioxidant under certain conditions. In fact, BHA appears to cause oxidation of susceptible moieties in certain formulations and/or under certain conditions due to the presence of free radicals. As such, it would be advantageous to find improved antioxidants or improved methods to formulate presently utilized antioxidants in an effort to enhance their efficacy.

Generally, BHA contains about 90 to about 93 percent of the major isomer 2-tert-butyl-4-methoxy-phenol. This isomer is crystallized in a double helical structure (Form I). Accordingly, it is an object of the present invention to provide a more effective antioxidant.

It is a further object of the invention to provide a process of preparing a more effective antioxidant.

### SUMMARY OF THE INVENTION

In a first embodiment, the present invention provides a novel polymorph (Form II) of 2-tert-butyl-4-methoxy-phenol, wherein the crystal packing occurs via the self-assembly of six molecules in a hexagonal geometry.

In another embodiment, the present invention provides a process for the preparation of 2-tert-butyl-4-methoxy-phenol form II, which comprises:
(a) obtaining pure 2-tert-butyl-4-methoxy-phenol; and
(b) crystallizing 2-tert-butyl-4-methoxy-phenol from an appropriate solvent under conditions which lead to the formation of 2-tert-butyl-4-methoxy-phenol form II.

The invention further provides for mixtures of 2-tert-butyl-4-methoxy-phenol which comprise form II. In another embodiment, a composition comprising at least about 1, 2, 3, 4, 5, 10, 15, 20, 25, 30, 35, 40, 45, 50, 55, 60, 65, 70, 75, 80, 85, 90, 95, or greater than about 99 percent form II is included.

In another embodiment, form II of 2-tert-butyl-4-methoxy-phenol is crystallized in a hydrocarbon solvent wherein the concentration of 2-tert-butyl-4-methoxy-phenol is at least about 150 mg/mL.

In another embodiment, the present invention provides a process for the preparation of 2-tert-butyl-4-methoxy-phenol form II, which comprises:
(a) obtaining BHA; and
(b) crystallizing 2-tert-butyl-4-methoxy-phenol from an appropriate solvent under conditions which lead to the formation of 2-tert-butyl-4-methoxy-phenol form II.

The invention further provides a method of improving the antioxidant properties of 2-tert-butyl-4-methoxy-phenol, which comprises:
(a) combining 2-tert-butyl-4-methoxy-phenol with a hydrocarbon solvent;
(b) crystallizing form II of 2-tert-butyl-4-methoxy-phenol from solution; and
(c) collecting the crystallized product.

In another embodiment, the additional step of combining said crystallized product to a formulation containing an oxidizable moiety is included.

In another embodiment, the present invention provides a method of crystallizing pure form II of 2-tert-butyl-4-methoxy-phenol from a mixture of form I and form II.

The invention further provides a method of improving the antioxidant properties of BHA, which comprises:
(a) combining BHA with a hydrocarbon solvent;
(b) crystallizing the BHA from solution; and
(c) collecting the crystallized product.

In another embodiment, the additional step of combining said crystallized product to a formulation containing an oxidizable moiety is included.

In one embodiment, the crystallized product consists of pure form II. In another embodiment, the crystallized product comprises a mixture of form II and minor isomer where the amount of form II is 50, 55, 60, 65, 70, 75, 80, 85, 90, 95 percent or more. In another embodiment, the crystallized product contains a mixture of form I, form II, and minor isomer where the amount of form II is 50, 55, 60, 65, 70, 75, 80, 85, 90, 95 percent or more.

The invention further provides a composition comprising the form II polymorph of 2-tert-butyl-4-methoxy-phenol. In one aspect, the composition is a pharmaceutical composition. In one embodiment, the pharmaceutical composition further comprises an active pharmaceutical ingredient (API) with one or more oxidizable functional groups including, but not limited to, amino, phenolic, hydroxyl amino, aldehyde, alkene, benzyl, isoxazole, mercapto, sulfone, or sulfoxide groups. The pharmaceutical composition may further comprise one or more pharmaceutically-acceptable carriers, diluents, or excipients. In another embodiment, the carriers, diluents, or excipients comprise one or more oxidizable functional groups including, but not limited to, amino, phenolic, hydroxyl amino, aldehyde, alkene, benzyl, isoxazole, mercapto, sulfone, or sulfoxide groups.

### BRIEF DESCRIPTION OF THE DRAWINGS

FIG. 1 Single crystal X-ray diffractogram of Form I

FIG. 2 DSC thermogram of Form I

FIG. 3 Single crystal X-ray diffractogram of Form II

FIG. 4 DSC thermogram of Form II

### DETAILED DESCRIPTION OF THE INVENTION

The terms “BHA” and “butylated hydroxy anisole” herein refer to a mixture of both the major isomer (2-tert-butyl-4-methoxy-phenol) and the minor isomer (3-tert-butyl-4-methoxy-phenol) as normally found in the commercially available product. The weight percent of major to minor isomer can vary, but generally falls within a range of about 80 to 95 percent (w/w) major isomer, for example, about 90 to 93 percent (w/w) major isomer.

The crystalline state of a compound can be unambiguously described by several crystallographic parameters: unit cell dimensions, space group, and atomic position of all atoms in the compound relative to the origin of its unit cell. These parameters are experimentally determined by single crystal x-ray analysis. It is possible for a compound to form more than one type of crystal. These different crystalline forms are called polymorphs. It has been discovered that there are two polymorphs of 2-tert-butyl-4-methoxy-phenol. This discovery was confirmed by two separate single crystal x-ray analyses. Form I is the major isomer in butylated hydroxy anisole (BHA). Form II is a novel polymorph whose properties are superior to those of form I as an antioxidant.

Single crystal x-ray analysis was employed in the characterization of form I. The crystal structure was determined to be two intertwining 4-fold helices. This polymorph (form I) assembles via OH . . . ether hydrogen bonds, head to tail. The distances between oxygen atoms in these hydrogen bonds (O . . . O) were determined to be about 2.707, 2.710, and 2.740 angstroms. This double helical structure is further characterized by a hydrophobic exterior surface due to the tert-butyl groups oriented outward. Such an open configuration allows the terminating ends of each helix to be susceptible to attack from free radicals. The discovery of form II of 2-tert-butyl-4-methoxy-phenol, which eliminates the risk of premature radical formation by virtue of its crystal structure, is a useful innovation.

The present invention provides a novel polymorph (Form II) of 2-tert-butyl-4-methoxy-phenol. Single crystal x-ray analysis was employed in the characterization of form II. The crystal structure was determined to be a closed hexamer. This polymorph (form II) assembles via OH . . . ether hydrogen bonds, head to tail, similar to form I. The distance between oxygen atoms in these hydrogen bonds (O . . . O) were determined to be about 2.778 angstroms. The closed nature of form II prevents the formation of radicals. Therefore, the present invention provides for the preparation of an antioxidant with a greatly reduced propensity for autooxidation.

2-tert-butyl-4-methoxy-phenol can be obtained via purification of BHA through recrystallization or it can be synthesized in pure form. In one embodiment, the preparation of form II can be completed by crystallizing 2-tert-butyl-4-methoxy-phenol from a hydrocarbon solvent. In another embodiment, the solvent is a mixture of solvents. In another embodiment, the solvent is warm (e.g., about 40 degrees C.) n-pentane or n-heptane. The concentration of 2-tert-butyl-4-methoxy-phenol in solution is typically about 50 to about 300 mg/mL, specifically about 100 to about 250 mg/mL, more specifically about 150 to about 200 mg/mL, for example, about 150 mg/mL.

Mixtures of form I and form II can also be used as superior antioxidants to pure form I. For example, a mixture with 95 percent (w/w) form II, or a mixture with 90 percent (w/w) form II, or mixtures with 85, 80, 75, 70, 65, 60, 55, 50, 40, 30, 20, or 10 percent (w/w) form II can be used to protect against oxidation. In another embodiment, mixtures of form II (2-tert-butyl-4-methoxy-phenol) and the minor isomer (3-tert-butyl-4-methoxy-phenol) can be employed as a superior antioxidant to BHA. For example, a mixture with 95 percent (w/w) form II, or a mixture with 90 percent (w/w) form II, or mixtures with 85, 80, 75, 70, 65, 60, 55, 50, 40, 30, 20, or 10 percent (w/w) form II can be used to protect against oxidation. In another embodiment, mixtures of form II, form I, and the minor isomer (3-tert-butyl-4-methoxy-phenol) can be employed as a superior antioxidant to BHA.

In another embodiment, the present invention includes compositions in which at least 10, 20, 30, 40, 50, 60, 70, 80, 90, 95, or 99 percent by weight (w/w) of the BHA is in the form of 2-tert-butyl-4-methoxy-phenol form II. In another embodiment, compositions comprising less than or equal to about 5, 4, 3, 2, or 1 percent by weight (w/w) BHA in the form of 2-tert-butyl-4-methoxy-phenol form II may be specifically excluded from the present invention.

The antioxidant properties of 2-tert-butyl-4-methoxy-phenol can be improved by converting some or all of the sample into form II crystals. This is achieved by mixing 2-tert-butyl-4-methoxy-phenol with a hydrocarbon solvent (e.g., n-pentane or n-heptane) so as to dissolve the solute at a concentration of at least about 150 mg/mL. Upon dissolution, form II can be crystallized from warm solvent and the crystals can be collected by any of several techniques known to one skilled in the art. In another embodiment, form II can be prepared via the addition of form II seed crystals to a collection of form I crystals. Form I crystals will spontaneously convert to form II crystals, aided by the high vapor pressure of 2-tert-butyl-4-methoxy-phenol. Such form II crystals can be added in appropriate amounts to an oxidizable formulation or moiety. An appropriate amount of antioxidant can vary, based upon the particular application, from 0.001 to 5 percent (w/w), or more specifically from 0.01 to 0.1 percent (w/w). The antioxidant activity of form II is commenced upon dissolution or conversion to an amorphous state in the presence of such oxidizable functional groups. The closed hexameric crystal structure of form II acts as a shield, keeping the molecules intact until the crystal structure is destroyed via dissolution or conversion to an amorphous state.

Many compositions include BHA or other antioxidants as preservatives or protective species. Commonly, pharmaceutical compositions and medicaments incorporate antioxidants as a means to prolong the shelf life of a drug. In another embodiment, form II is employed as an antioxidant in a pharmaceutical composition or a medicament comprising one or more ingredients susceptible to oxidation. BHA is commonly used as a food additive in an effort to prolong the shelf life of many perishable foods (e.g., vegetables, fruits, etc.). In another embodiment, form II is used as a food preservative (e.g., spray, coating). Antioxidants are also used in polymer structures. In another embodiment, form II is employed as an antioxidant in polymeric structures and blends. In another embodiment, form II is used as a preservative in cosmetics, rubber, and petroleum products.

The crystalline compounds of the present invention exhibit significantly less oxidative activity relative to the same compositions without similarly processed 2-tert-butyl-4-methoxy-phenol.

The present invention will be further described by exemplification, but this description does not limit the scope of the invention.

Analytical Methods

DSC analysis of the samples was performed using a Q1000 Differential Scanning Calorimeter (TA Instruments, New Castle, Del., U.S.A.), which uses Advantage for QW-Series, version 1.0.0.78, Thermal Advantage Release 2.0 (2001 TA Instruments-Water LLC). In addition, the analysis software used was Universal Analysis 2000 for Windows 95/95/2000/NT, version 3.1E;Build 3.1.0.40 (2001 TA Instruments-Water LLC).

For the DSC analysis, the purge gas used was dry nitrogen, the reference material was an empty aluminum pan that was crimped, and the sample purge was 50 mL/minute.

DSC analysis of the sample was performed by placing the sample in an aluminum pan with a crimped pan closure. The starting temperature was typically 20° C. with a heating rate of 10° C./minute, and the ending temperature was 200° C.

A single-crystal X-ray diffraction pattern for the samples was obtained using a D/Max Rapid, Contact (Rigaku/MSC, The Woodlands, Tex., U.S.A.), which uses as its control software RINT Rapid Control Software, Rigaku Rapid/XRD, version 1.0.0 (1999 Rigaku Co.). In addition, the analysis software used were RINT Rapid display software, version 1.18 (Rigaku/MSC), and JADE XRD Pattern Processing, versions 5.0 and 6.0 ((1995-2002, Materials Data, Inc.).

For the single-crystal XRD analysis, the acquisition parameters were as follows: source was Cu with a K line at 1.5406 Å; x-y stage was manual; collimator size was 0.3 mm; capillary tube (Charles Supper Company, Natick, Mass., U.S.A.) was 0.3 mm ID; reflection mode was used; the power to the X-ray tube was 46 kV; the current to the X-ray tube was 40 mA; the omega-axis was oscillating in a range of 0-5 degrees at a speed of 1 degree/minute; the phi-axis was spinning at an angle of 360 degrees at a speed of 2 degrees/second; 0.3 mm collimator; the collection time was 60 minutes; the temperature was room temperature; and the heater was not used. The sample was presented to the X-ray source in a boron rich glass capillary.

In addition, the analysis parameters were as follows: the integration 2-theta range was 2-60 degrees; the integration chi range was 0-360 degrees; the number of chi segments was 1; the step size used was 0.02; the integration utility was cylint; normalization was used; dark counts were 8; omega offset was 180; and chi and phi offsets were 0.

The relative intensity of peaks in a diffractogram is not necessarily a limitation of the single-crystal XRD pattern because peak intensity can vary from sample to sample, e.g., due to crystalline impurities. Further, the angles of each peak can vary by about +/−0.1 degrees, preferably +/−0.05. The entire pattern or most of the pattern peaks may also shift by about +/−0.1 degree due to differences in calibration, settings, and other variations from instrument to instrument and from operator to operator. All reported single-crystal XRD peaks in the Figures, Examples, and elsewhere herein are reported with an error of about ±0.1 degrees 2-theta.

Single crystal x-ray data were collected on a Bruker SMART-APEX CCD diffractometer (M. J. Zaworotko, Department of Chemistry, University of South Florida). Lattice parameters were determined from least squares analysis. Reflection data was integrated using the program SAINT. The structure was solved by direct methods and refined by full matrix least squares using the program SHELXTL (Sheldrick, G. M. SHELXTL, Release 5.03; Siemens Analytical X-ray Instruments Inc.: Madison, Wis.).

For single-crystal XRD data herein, both polymorphs of the present invention may be characterized by any one, any two, any three, any four, any five, any six, any seven, any eight or more of the 2-theta angle peaks. Any one, two, three, four, five, or six DSC transitions can also be used to characterize the polymorphs of the present invention. The different combinations of the single-crystal XRD peaks and the DSC transitions can also be used to characterize the polymorphs. Single-crystal x-ray diffraction data can also be used to characterize the polymorphs.

### EXAMPLE 1

Isolation of Major Isomer of BHA

BHA consists of the major isomer 2-tert-butyl-4-methoxy-phenol and the minor isomer 3-tert-butyl-4-methoxy-phenol. All polymorphism/crystallization studies were completed on 2-tert-butyl-4-methoxy-phenol after isolation from the minor isomer. The isolation was completed by crystallization from warm n-heptane. Other hydrocarbon solvents, such as n-pentane, were also used to isolate 2-tert-butyl-4-methoxy-phenol.

### EXAMPLE 2

Single Crystal X-Ray Diffraction of Form I

Upon obtaining pure 2-tert-butyl-4-methoxy-phenol, crystals were prepared for single crystal XRD analysis. Form I was made by crystallization from n-heptane and n-pentane. Form I crystals were formed from solutions that were more dilute (less than 150 mg/mL 2-tert-butyl-4-methoxy-phenol) than those which resulted in form II. Long rods were obtained for single crystal x-ray analysis by the slow evaporation of solvent. In the vial, the form may appear like a ball of cotton or like discrete rods, depending on the crystallization conditions (e.g., concentration, temperature, etc.). Form I was found to have an endothermic transition at about 61 degrees C. and the calculated density was 1.158 g/cm³.

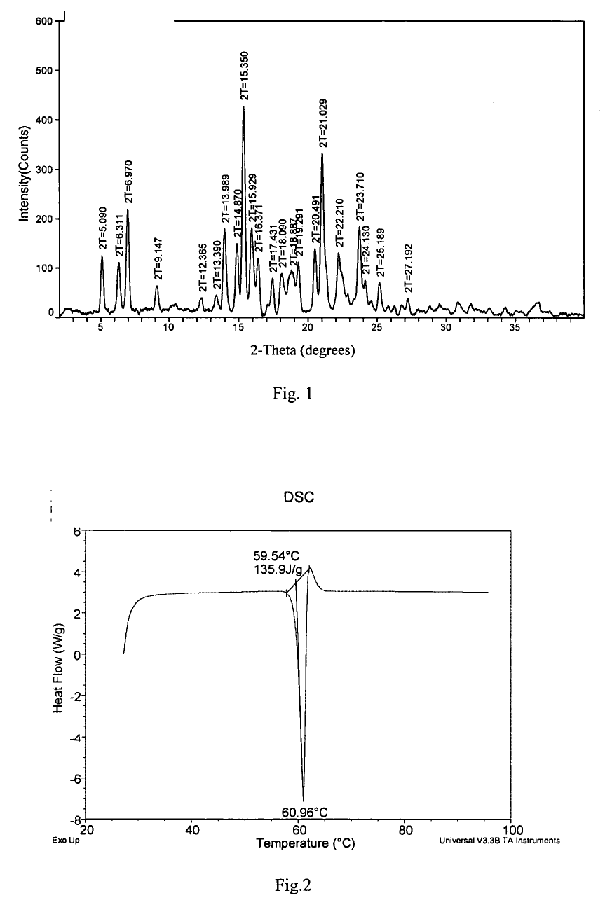

The single crystal XRD diffractogram has peaks that can be used to characterize the crystal structure of form I, and include any one or any combination comprising any two, any three, any four, any five, any six, any seven, or more than seven 2-theta angle peaks of 5.09, 6.31, 6.97, 13.99, 14.87, 15.35, 15.93, 16.37, 17.43, 18.09, 18.89, 19.29, 20.49, 21.03, 22.21, and 23.71 degrees or any other peaks in FIG. 1.

Single crystal data for Form I: C₁₁H₁₆O₂, M=180.24, triclinic, space group P-1; a=6.3179(11) angstroms, b=14.364(3) angstroms, c=17.960(3) angstroms, α=74.636(3) degrees, β=80.608(4) degrees, γ=86.767(3) degrees, V=1550.5(5) cubic angstroms, T=100(2) K, Z=6, μ(Mo—Kα)=0.078 mm⁻¹, D_{c}=1.158 Mg m⁻³, λ=0.71073 Å, F(000)=588, 2θₘₐₓ=28.24 degrees, 9728 reflections measured, 6805 unique (Rᵢₙₜ=0.0320). Final residuals for 352 parameters were R₁=0.0547, wR₂=0.1403 for I>2σ(I), and R₁=0.0871, wR₂=0.1618 for all 6805 data.

Differential Scanning Calorimetry of Form I

2.0760 mg of collected sample was placed into a crimped aluminum DSC pan with a cover. Results of the DSC thermogram (FIG. 2) showed an endothermic transition at about 61 degrees C. with an intensity of about 136 J/g.

### EXAMPLE 3

Preparation of Form II Polymorph

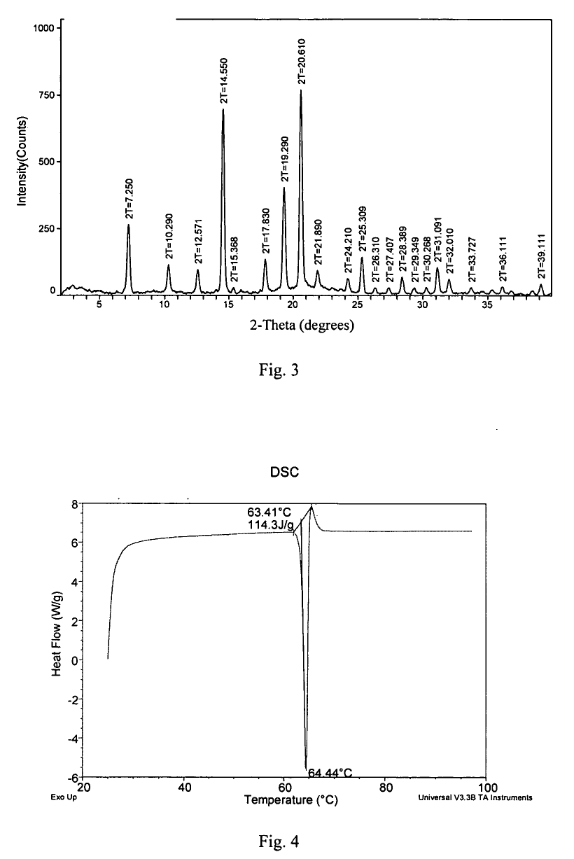

A solution of 2-tert-butyl-4-methoxy-phenol in n-pentane was prepared at a concentration of about 150 mg/mL and heated to about 40 degrees C. The solution was then allowed to cool. Upon cooling, crystallization occurred in the form of large chunks of solid. The resultant crystals showed fibers growing out of the chunks upon view with an optical microscope (50× magnification). However, the single crystal XRD (FIG. 3) is consistent with pure form II, which suggests that form II can exist in a fiber like morphology similar to that observed for form I. Form II was found to have an endothermic transition at about 64 degrees C. and the calculated density is 1.136 g/cm³.

The single crystal XRD diffractogram has peaks that can be used to characterize the crystal structure of form II, and include any one or any combination comprising any two, any three, any four, any five, any six, any seven, or more than seven 2-theta angle peaks of 7.25, 10.29, 12.57, 14.55, 17.83, 19.29, 20.61, 21.89, 25.31, 28.39, and 31.09 degrees or any other peaks in FIG. 3.

Single crystal data for Form II: C₁₁H₁₆O₂, M=180.24, trigonal, space group R-3; a=24.2612(11) angstroms, b=24.2612(11) angstroms, c=9.3049(8) angstroms, γ=120 degrees, V=4743.1(5) cubic angstroms, T=100(2) K, Z=18, μ(Mo—Kα)=0.076 mm⁻¹, D_{c}=1.136 Mg m⁻³, λ=0.71073 Å, F(000)=1764, 2θₘₐₓ=28.30 degrees, 10131 reflections measured, 2504 unique (Rᵢₙₜ=0.0600). Final residuals for 130 parameters were R₁=0.0435, wR₂=0.1185 for I>2σ(I), and R₁=0.0552, wR₂=0.1269 for all 2504 data.

Differential Scanning Calorimetry of Form II

1.0540 mg of collected sample was placed into a crimped aluminum DSC pan with a cover. Results of the DSC thermogram (FIG. 4) show an endothermic transition at about 64 degrees C. with an intensity of about 114 J/g.

1. A composition comprising 2-tert-butyl-4-methoxy-phenol form II.

2. The composition of claim 1, further comprising 2-tert-butyl-4-methoxy-phenol form I.

3. The composition of claim 1, further comprising 3-tert-butyl-4-methoxy-phenol.

4. The composition of claim 1, further comprising 2-tert-butyl-4-methoxy-phenol form I and 3-tert-butyl-4-methoxy-phenol.

5. The composition of claim 1, wherein the composition is a pharmaceutical composition.

6. The composition of claim 1, wherein the composition is a polymer structure or blend.

7. The composition of claim 1, wherein the composition is a food preservative.

8. A polymorph of 2-tert-butyl-4-methoxy-phenol, wherein:
(a) said polymorph is characterized by a single-crystal X-ray diffraction pattern comprising peaks expressed in terms of 2 theta angles, wherein:
(i) said X-ray diffraction pattern comprises peaks at 7.25, 14.55, and 19.29;
(ii) said X-ray diffraction pattern comprises peaks at 7.25, 10.29, and 17.83;
(iii) said X-ray diffraction pattern comprises peaks at 14.55, 19.29, and 20.61;
(iv) said X-ray diffraction pattern comprises peaks at 7.25 and 14.55;
(v) said X-ray diffraction pattern comprises peaks at 19.29 and 20.61;
(vi) said X-ray diffraction pattern comprises a peak at 7.25;
(vii) said X-ray diffraction pattern comprises a peak at 14.55;
(viii) said X-ray diffraction pattern comprises a peak at 20.61;
(ix) said X-ray diffraction pattern comprises peaks at 7.25, 10.29, 14.55, and 20.61; or
(x) said X-ray diffraction pattern comprises peaks at 7.25, 14.55, 19.29, 20.61, and 25.31;
(b) said polymorph is characterized by single-crystal X-ray parameters, wherein:
(i) a=24.2612(11) angstroms, b=24.2612(11) angstroms, c=9.3049(8) angstroms, γ=120 degrees;
(ii) space group R-3; a=24.2612(11) angstroms, b=24.2612(11) angstroms, c=9.3049(8) angstroms, γ=120 degrees, V=4743.1(5) cubic angstroms; or
(iii) space group R-3; a=24.2612(11) angstroms, b=24.2612(11) angstroms, c=9.3049(8) angstroms, γ=120 degrees, V=4743.1(5) cubic angstroms, Z=18, D_{c}=1.136 Mg m⁻³; or
(c) said polymorph is characterized by an endothermic transition at about 64 degrees C., observed by DSC analysis.

9. A composition consisting of 2-tert-butyl-4-methoxy-phenol form II.

10. A process for the preparation of 2-tert-butyl-4-methoxy-phenol form II, comprising:
(a) obtaining pure 2-tert-butyl-4-methoxy-phenol; and
(b) crystallizing 2-tert-butyl-4-methoxy-phenol from an appropriate solvent under conditions which lead to the formation of 2-tert-butyl-4-methoxy-phenol form II.

11. The process of claim 10, wherein said solvent is n-heptane or n-pentane.

12. The process of claim 10, wherein said solvent is warm n-heptane or warm n-pentane.

13. The process of claim 10, wherein said conditions comprise a concentration of 2-tert-butyl-4-methoxy-phenol of at least about 150 mg/mL.

14. A process for the preparation of 2-tert-butyl-4-methoxy-phenol form II, comprising:
(a) obtaining BHA; and
(b) crystallizing 2-tert-butyl-4-methoxy-phenol from an appropriate solvent under conditions which lead to the formation of 2-tert-butyl-4-methoxy-phenol form II.

15. A process of improving the antioxidant properties of 2-tert-butyl-4-methoxy-phenol, comprising:
(a) combining 2-tert-butyl-4-methoxy-phenol with a hydrocarbon solvent;
(b) crystallizing form II of 2-tert-butyl-4-methoxy-phenol from solution; and
(c) collecting the crystallized product.

16. The process of claim 15, further comprising adding said crystallized product to a formulation containing an oxidizable moiety.

17. A process of improving the antioxidant properties of BHA, comprising:
(a) combining BHA with a hydrocarbon solvent;
(b) crystallizing the BHA from solution; and
(c) collecting the crystallized product.

18. The process of claim 17, further comprising adding said crystallized product to a formulation containing an oxidizable moiety.
